(19) United States
(12) Patent Application Publication
(10) Pub. No.: US 2025/0276141 A1
(43) Pub. Date: Sep. 4, 2025

(54) RESPIRATORY THERAPY DEVICE

(71) Applicant: Loewenstein Medical Technology S.A., Luxembourg (LU)

(72) Inventors: Angela GERLACH, Hamburg (DE); Kathleen VEERS, Kiel (DE); Alexander OSTER, Gettorf (DE)

(21) Appl. No.: 19/062,329

(22) Filed: Feb. 25, 2025

(30) Foreign Application Priority Data
Mar. 1, 2024 (DE) 10 2024 105 940.0

(51) Int. Cl. A61M 16/00

(52) U.S. Cl. CPC A61M 16/0066; A61M 2202/0007; A61M 2202/02; A61M 2205/42; A61M 2205/7536; A61M 2205/7545

(57) ABSTRACT

A respiratory therapy device comprises a gas inlet, a gas outlet, a fan for conveying respiratory gas from the gas inlet to the gas outlet and a chamber for sound damping. The chamber comprises a first opening, a second opening and a tubular channel, which comprises an inner channel section protruding from the first opening into the interior of the chamber and/or an outer channel section protruding from the first opening into the exterior surroundings of the chamber. The device is configured so that the respiratory gas, when it is conveyed from the gas inlet to the gas outlet, flows between the first opening and the second opening through the chamber and passes the channel in this case.

### CROSS-REFERENCE TO RELATED APPLICATIONS

The present application claims priority under 35 U.S.C. § 119 of German Patent Application No. 10 2024 105 940.0, filed Mar. 1, 2024, the entire disclosure of which is expressly incorporated by reference herein.

### BACKGROUND OF THE INVENTION

### 1. Field of the Invention

The invention relates to a respiratory therapy device.

### 2. Discussion of Background Information

A respiratory therapy device can be used for treating sleep-related breathing disorders, for example, an obstructive sleep apnea syndrome, or in the event of impairments of the respiratory pump, for example, due to a chronic obstructive pulmonary disorder (COPD). For this purpose, the respiratory therapy device can generate, for example, a continuous positive pressure in the airway of the patient, also called CPAP (continuous positive airway pressure). Such a respiratory therapy device generally comprises a fan, which can cause clearly audible flow noises depending on the speed. Such noises can be perceived as unpleasant or even as disturbing, in particular when sleeping.

In view of the foregoing, it would be advantageous to have available a respiratory therapy device whose noises can be effectively damped in operation, so that they cannot be perceived at all or at least are no longer perceived as unpleasant or disturbing.

### SUMMARY OF THE INVENTION

The invention provides to a respiratory therapy device which comprises a gas inlet, a gas outlet, a fan for conveying respiratory gas from the gas inlet to the gas outlet, and a chamber for sound damping. The chamber comprises a first opening, a second opening, and a tubular channel, which comprises an inner channel section protruding from the first opening into the interior of the chamber and/or an outer channel section protruding from the first opening into exterior surroundings of the chamber. The respiratory therapy device is designed so that the respiratory gas, when it is conveyed from the gas inlet to the gas outlet, flows between the first opening and the second opening through the chamber and in this case passes the channel, i.e., the inner channel section and/or the outer channel section.

Such a respiratory therapy device has the advantage that unpleasant or disturbing operating noises are significantly attenuated due to the special sound damping chamber, in particular without special foam insulation having to be integrated into the device for this purpose. This can simplify the production, maintenance, and cleaning of the device.

A “respiratory therapy device” can be understood in general as a ventilator for invasive or non-invasive ventilation or a device for secretion removal, for example, in the form of a coughing machine. The respiratory therapy device can be suitable in particular for a high-flow therapy. A “respiratory therapy device” can also be understood as one component (of multiple components) of a ventilator or a ventilation system comprising a ventilator.

The gas inlet and the gas outlet can be connected to one another via a flow path within the respiratory therapy device. The flow path can comprise, for example, at least one of the following components: the channel, the chamber, the fan, a gas line, an air humidifier, a gas sensor. The gas inlet can be connectable to a suitable respiratory gas source and/or external surroundings of the respiratory therapy device. The gas outlet can be connectable to a hose for supplying a patient with the respiratory gas. The hose can be connected, for example, to at least one of the following patient interfaces: a tube, a nose mask, a nasal cannula, a facial mask. The gas inlet and/or the gas outlet can be formed, for example, in an (exterior) housing of the respiratory therapy device.

A “fan” can be understood in general as a turbomachine for applying a pressure to the respiratory gas. The fan can generate, for example, a pressure ratio of from about 1.0 to about 1.3, of from about 1.3 to about 3.0, or of greater than about 3.0 (each from the pressure side to the suction side).

The chamber can generally be understood as a spring-mass system, which can damp sound in relevant frequency ranges depending on its tuning, in particular depending on a length and/or a flow cross section of the inner channel section and/or the outer channel section. The interior of the chamber can be a cavity delimited by multiple walls—for example, in a length direction and/or a height direction and/or a width direction. The fan can be fluidically coupled directly and/or indirectly, for example, by means of one or more gas lines, via its (suction-side) inlet and/or its (pressure-side) outlet with the first opening and/or the second opening. For example, a first gas line can connect, on the one hand, the first opening to the inlet of the fan and, on the other hand, the second opening to the gas inlet, wherein a second gas line can connect the outlet of the fan to the gas outlet. Alternatively, a first gas line can connect, on the one hand, the gas inlet to the inlet of the fan and, on the other hand, the second opening to the outlet of the fan, wherein a second gas line can connect the first opening to the gas outlet. Both embodiments have the effect that the respiratory gas flows from the second opening to the first opening through the chamber when the fan is activated. However, an embodiment is also possible in which the respiratory gas flows from the first opening to the second opening when the fan is activated. With regard to the sound-damping effect it is proven to be particularly advantageous if the chamber is arranged on the suction side of the fan, i.e. before the inlet of the fan viewed in the flow direction of the respiratory gas.

A “tubular channel” can be expediently understood above and hereinafter as a channel having an at least partially closed cross-sectional shape, for example, in contrast to a trough-shaped channel having an open cross-sectional shape. The cross section of the tubular channel can be, for example, circular, ellipsoidal, or cuboid. However, more complex cross-sectional shapes are also possible, such as a L-shape or T-shape and/or cross-sectional shapes varying with respect to the longitudinal direction of the tubular channel. The tubular channel can be designed, for example, as a pipe and/or hose.

A “gas line” can be understood, for example, as a (rigid) pipe, a nozzle, a hose, or a combination made up of at least two of these examples.

An “opening” as in “first opening” or “second opening” can generally be understood as a passage through a wall of the chamber. The passage can in particular connect an inner surface of the wall facing toward the interior of the chamber to an outer surface of the wall facing toward the exterior surroundings of the chamber.

The first opening and the second opening can be at least partially opposite to one another viewed in the direction of a flow of the respiratory gas through the chamber, i.e. can partially or completely overlap. Alternatively, the first opening and the second opening can be arranged offset in relation to one another.

It is possible that the first opening forms the gas inlet and/or the second opening forms the gas outlet. Alternatively, the first opening can form the gas outlet and/or the second opening can form the gas inlet. In other words, the gas inlet and/or the gas outlet can be formed by one or more openings or passages in one or more walls of the chamber. Pressure losses can thus be significantly reduced in comparison to an embodiment in which the chamber is connected via one or more gas lines to the gas inlet and/or the gas outlet. This can make the respiratory therapy device more efficient and/or quieter in operation.

A “channel section” as in “inner channel section” or “outer channel section” can be understood, for example, as a tubular piece, which protrudes perpendicularly or obliquely from a wall of the chamber and is connected on one or both sides. The inner channel section can be separated, for example, by an air gap from the second opening and/or a wall of the chamber, in particular a wall of the chamber opposite to the first opening. The channel or at least one of the channel sections can extend straight and/or curved viewed in its longitudinal direction. A curved channel (section) can cause the respiratory gas flow to strike the channel wall not perpendicularly, but rather more or less obliquely, which can reduce noises.

The inner channel section can substantially correspond in its length with the outer channel section or can deviate significantly from the outer channel section, for example, by at least about 10%, at least about 30%, or at least about 50%.

Various embodiments of the invention are described hereinafter. These embodiments are not to be understood as a restriction of the scope of the invention.

According to one embodiment, the tubular channel, more precisely its inner cavity, can be delimited at least in some sections by a wall section of the chamber and/or another housing of the respiratory therapy device, for example, its exterior housing.

According to one embodiment, the tubular channel can be delimited at least in some sections by at least one plug-in part connectable in a friction-locked and/or formfitting manner with a housing of the respiratory therapy device without the aid of a tool. In this case, the tubular channel can be formed at least in some sections by a corresponding gap between the housing and the plug-in part (or the plug-in parts). This can facilitate the cleaning of the respiratory therapy device.

According to one embodiment, the second opening can be at least partially opposite to the first opening and/or an open end of the inner channel section viewed in a direction of flow of the respiratory gas through the chamber. The term “open end” can be understood above and hereinafter in particular as an unconnected end.

In more general terms, at least one of the openings of the tubular channel can be arranged with a certain offset relative to at least one of the openings of the chamber, preferably an inlet opening of the chamber, so that the respective openings partially (or completely) overlap. This has the effect that under certain circumstances fewer soundwaves penetrate directly through the chamber.

According to one embodiment, a (for example, averaged) ratio R=P²/A can be from about 14 to about 30, from about 16 to about 30, or from about 20 to about 30, wherein P stands for a (for example, averaged) circumference of the tubular channel and A stands for a (for example, averaged) cross-sectional area of the tubular channel. Using such R values—more precisely due to the accompanying significant increase of the frictional surface for a given cross section in comparison to other R values—it was possible to achieve a particularly effective sound damping in experiments. In certain cases, a R value less than about 14 (for example about 12) or greater than about 30 (for example about 40) is also conceivable.

An embodiment has also proven to be particularly favorable in which the (for example averaged) flow cross section of the chamber at least in a section between one of the openings of the chamber and one of the openings of the tubular channel is twice as wide or more than twice as wide as the (for example averaged) flow cross section of the tubular channel.

According to one embodiment, the respiratory therapy device can be designed so that the respiratory gas, when it is conveyed from the gas inlet to the gas outlet, flows through the chamber from the second opening to the first opening and passes the channel in this case.

According to one embodiment, the second opening can have a different flow cross section, in particular a significantly larger flow cross section, than the first opening and/or than an open end of the inner channel section. The second opening can in particular have a significantly larger flow cross section than the first opening and/or than the open end of the inner channel section if the respiratory gas-when it is conveyed from the gas inlet to the gas outlet-flows from the second opening to the first opening through the chamber. Vice versa, the second opening can in particular have a significantly smaller flow cross section than the first opening and/or than the open end of the inner channel section if the respiratory gas-when it is conveyed from the gas inlet to the gas outlet-flows from the first opening to the second opening through the chamber. Alternatively, the flow cross section of the second opening can substantially correspond in its size and/or shape with the flow cross section of the first opening and/or the open end of the inner channel section.

According to one embodiment, the second opening can terminate flush with an inner surface of a wall of the chamber facing toward the interior of the chamber or can protrude from the inner surface to an extent which is not noteworthy. Alternatively or additionally, the second opening can terminate flush with an outer surface of a wall of the chamber facing toward the exterior surroundings of the chamber or can protrude from the outer surface to an extent which is not noteworthy.

Furthermore, it is possible that the first opening terminates flush on one side with an inner surface or an outer surface of a wall of the chamber or protrudes from the inner or outer surface to an extent which is not noteworthy.

Such a one-sided or two-sided flush terminus of the first or second opening has the advantage that pressure losses can be reduced due to the flow directed in the same direction.

According to one embodiment, a flow cross section of the inner channel section can substantially correspond in its size and/or shape with a flow cross section of the outer channel section.

According to one embodiment, the inner channel section and the outer channel section can have a common longitudinal axis. The common longitudinal axis of the inner channel section and the outer channel section can extend straight and/or curved. Alternatively, the inner channel section and the outer channel section can have longitudinal axes parallel to one another.

According to one embodiment, the chamber can be delimited in a longitudinal direction, on the one hand, by a first wall and, on the other hand, by a second wall. In this case, the first wall can have the first opening and/or the second wall can have the second opening. In other words, the first opening and the second opening can be arranged on sides of the chamber opposite to one another. “Longitudinal direction” can be understood in general as a first, for example, horizontal or vertical spatial direction in a three-dimensional coordinate system. “Wall” as in “first wall” or “second wall” can be understood, for example, as at least one section of a bottom, a cover, a side wall, or a chamber cover of the chamber or a combination of at least two of these sections. For example, the first wall can be integrally formed with the inner channel section and/or the outer channel section, for example, in an injection molding method.

According to one embodiment, the inner channel section can protrude into the interior of the chamber at most up to half of a (total) length of the chamber in the longitudinal direction between the first wall and the second wall. The length of the chamber can be, for example, from about 30 mm to about 50 mm, preferably from about 35 mm to about 45 mm. Alternatively or additionally, a (total) height of the chamber in a height direction orthogonal to the longitudinal direction can be from about 20 mm to about 40 mm, preferably from about 25 mm to about 35 mm, and/or a (total) width of the chamber in a width direction orthogonal to the length direction and to the height direction can be from about 30 mm to about 50 mm, preferably from about 35 mm to about 45 mm.

According to one embodiment, a first end of the inner channel section can be connected to the first wall and a second unconnected end of the inner channel section can protrude into the interior of the chamber. The second unconnected end of the inner channel section can additionally be open.

In a corresponding manner, a first end of the outer channel section can be connected to the first wall and a second connected or unconnected end of the outer channel section can protrude into the exterior surroundings of the chamber. For example, the second end of the outer channel section can be connected directly or indirectly, for example, by means of a nozzle and/or a pipe and/or a hose, to at least one of the following components of the respiratory therapy device: the gas inlet, the gas outlet, the inlet of the fan, the output of the fan, an additional chamber for sound damping, in particular an additional chamber as is described in more detail hereinafter.

According to one embodiment, the chamber can furthermore comprise a chamber cover, which is removable and/or movably mounted, for example, rotatable and/or displaceable, for closing the chamber. This facilitates the access to the interior of the chamber, for example, for maintenance, cleaning, or repair purposes.

According to one embodiment, the chamber cover, when it closes the chamber, can form at least one section of the first wall and/or the second wall.

According to one embodiment, the chamber can furthermore comprise a filter material for filtering particles and/or moisture from the respiratory gas flowing between the first opening and the second opening through the chamber. The filter material can be arranged in the operational state of the respiratory therapy device at least partially in a flow path of the respiratory gas between the first opening and the second opening through the chamber, so that the respiratory gas can flow through the filter material. The filter material can in particular be a porous material. For example, the filter material can comprise a foam, a sintered metal, a wire mesh, fibers, clay, or a combination of at least two of these examples. Further examples of suitable filter materials are polyester nonwoven materials, mixed artificial fibers in a propylene carrier, or synthetic polyester blends. Alternatively or additionally, the filter material can have special chemical and/or physical properties, due to which the filter material additionally has a strong sound-damping effect. The sound-damping effect of the chamber can thus be further improved. The filter material can be applied, for example, to a carrier to stabilize the filter material. The carrier itself can also be gas-permeable. For example, the carrier can be formed as a grid and/or net structure. Alternatively or additionally, the chamber can have at least one support element for supporting the filter material and/or the carrier on its inner wall, for example, in the form of a projection or a catch element.

According to one embodiment, the filter material can at least partially fill the interior of the chamber and/or can at least partially fill the channel. In particular, the filter material can largely or completely fill the interior of the chamber and/or the channel. This enables efficient filtering without the structural form of the chamber and/or the respiratory therapy device having to be adapted in a noticeable manner.

According to one embodiment, the filter material can be arranged opposite to the first opening and/or the second opening. In this case, the filter material can touch the respective opening, for example, cover it in an air-permeable manner, or can be separated from the respective opening by an air gap.

According to one embodiment, the filter material can be formed as part of an insert element insertable into the chamber and/or the channel. This enables easy replacement of the filter material. For example, the insert element can be formed as part of the chamber cover or vice versa.

According to one embodiment, the filter material can be fastened on the chamber cover and can be removable and/or movably mounted together with the chamber cover. The filter material can be fastened on the chamber cover so that it is located in the flow path of the respiratory gas when the chamber cover closes the chamber. This facilitates access to the interior of the chamber, for example, for maintenance, cleaning, or repair purposes. Moreover, this enables easy replacement of the filter material.

According to one embodiment, the chamber can furthermore comprise a further tubular channel. The further channel can comprise a further inner channel section protruding from the second opening into the interior of the chamber and/or a further outer channel section protruding from the second opening into the exterior surroundings of the chamber. In this case, the respiratory therapy device can be designed so that the respiratory gas furthermore passes the further channel when it is conveyed from the gas inlet to the gas outlet. The further inner channel section can substantially correspond in its length with the further outer channel section or can deviate decisively, for example, by at least about 10%, at least about 30%, or at least about 50%, from the further outer channel section.

The channel can substantially correspond in its length with the further channel or can deviate decisively, for example, by at least about 10%, at least about 30%, or at least about 50%, from the further channel.

According to one embodiment, a first end of the further inner channel section can be connected to the second wall and a second unconnected end of the further inner channel section can protrude into the interior of the chamber. The second unconnected end of the further inner channel section can additionally be open. For example, the second wall can be formed integrally with the further inner channel section and/or the further outer channel section, for example, in an injection molding method.

In a corresponding manner, a first end of the further outer channel section can be connected to the second wall and a second connected or unconnected end of the further outer channel section can protrude into the exterior surroundings of the chamber. For example, the second end of the further outer channel section can be connected directly or indirectly, for example, by means of a nozzle and/or a pipe and/or a hose, to at least one of the following components of the respiratory therapy device: the gas inlet, the gas outlet, the inlet of the fan, the output of the fan, an additional chamber for sound damping, in particular an additional chamber as is described in more detail hereinafter.

According to one embodiment, an open end of the further inner channel section of the first opening and/or an open end of the inner channel section can be at least partially opposite to one another viewed in the direction of a flow of the respiratory gas through the chamber.

According to one embodiment, an open end of the further inner channel section can have a different flow cross section, in particular a significantly larger flow cross section, than the first opening and/or than an open end of the inner channel section. The open end of the further inner channel section can in particular have a significantly larger flow cross section than the first opening and/or than the open end of the inner channel section if the respiratory gas-when it is conveyed from the gas inlet to the gas outlet-flows from the second opening to the first opening through the chamber. Vice versa, the open end of the further inner channel section can in particular have a significantly smaller flow cross section than the first opening and/or than the open end of the inner channel section if the respiratory gas-when it is conveyed from the gas inlet to the gas outlet-flows from the first opening to the second opening through the chamber. Alternatively, the flow cross section of the open end of the further inner channel section can substantially correspond in its size and/or shape with the flow cross section of the first opening and/or the open end of the inner channel section.

According to one embodiment, a length of the further inner channel section can be at most a third of a length of the inner channel section. A “length” can be understood as an extent of the respective channel section in its longitudinal direction, i.e. in the direction of its longitudinal axis.

According to one embodiment, the further inner channel section and the further outer channel section can have a common longitudinal axis. The common longitudinal axis of the further inner channel section and/or the further outer channel section can extend straight and/or curved.

According to one embodiment, a flow cross section of the further inner channel section can substantially correspond in its size and/or shape with a flow cross section of the further outer channel section. Alternatively, the flow cross section of the further inner channel section can decisively deviate in its size and/or shape from the flow cross section of the further outer channel section.

According to one embodiment, the channel can have a different flow cross section, in particular a significantly smaller flow cross section, than the further channel. The channel can in particular have a significantly smaller flow cross section than the further channel if the respiratory gas-when it is conveyed from the gas inlet to the gas outlet-flows from the second opening to the first opening through the chamber. Vice versa, the channel can in particular have a significantly larger flow cross section than the further channel if the respiratory gas-when it is conveyed from the gas inlet to the gas outlet-flows from the first opening to the second opening through the chamber.

According to one embodiment, a length of the further channel can be at most a third of a length of the channel. A “length” can be understood as an extension of the respective channel in its longitudinal direction, i.e. in the direction of its longitudinal axis.

According to one embodiment, the channel and the further channel can have a common longitudinal axis. The common longitudinal axis of the channel and the further channel can extend straight and/or curved.

According to one embodiment, a volume of the chamber can be from about 18 cm³ to about 160 cm³, in particular from about 30 cm³ to about 70 cm³.

According to one embodiment, a volume of the channel and/or the inner channel section can be from about 5 cm³ to about 12 cm³. The channel can substantially correspond in its volume with the further channel or can deviate decisively from the further channel, for example, by at least about 10%, at least about 30%, or at least about 50%. The inner channel section can substantially correspond in its volume with the further inner channel section or can decisively deviate from the further inner channel section, for example, by at least about 10%, at least about 30%, or at least about 50%.

According to one embodiment, a largest cross-sectional area of the chamber can be from about 8 cm² to about 16 cm², in particular from about 10 cm² to about 14 cm².

According to one embodiment, a largest cross-sectional area of the channel and/or the inner channel section can be from about 1.8 cm² to about 2.0 cm². The channel can substantially correspond in its largest cross-sectional area with the further channel or can deviate decisively from the further channel, for example, by at least about 10%, at least about 30%, or at least about 50%. The inner channel section can substantially correspond in its largest cross-sectional area with the further inner channel section or can deviate decisively from the further inner channel section, for example, by at least about 10%, at least about 30%, or at least about 50%.

Using such measures, it was possible to achieve particularly good results with respect to the sound-damping effect in experiments.

According to one embodiment, the respiratory therapy device can furthermore comprise an additional chamber for sound damping. Similarly as the chamber described above and hereinafter, the additional chamber can comprise an additional first opening, an additional second opening, and an additional tubular channel, wherein the additional channel can comprise an additional inner channel section protruding from the additional first opening into the interior of the additional chamber and/or an additional outer channel section protruding from the additional first opening into the exterior surroundings of the additional chamber. In this case, the respiratory therapy device can be designed so that the respiratory gas, when it is conveyed from the gas inlet to the gas outlet, furthermore flows between the additional first opening and the additional second opening through the additional chamber and passes the additional channel in this case.

According to one embodiment, the chamber can be connected in series to the additional chamber or the additional chambers, so that the respiratory gas flows in succession through the chambers connected to one another in series when it is conveyed from the gas inlet to the gas outlet.

The chamber can be fluidically connected, for example, via a passage in a partition wall and/or via a tubular connection channel to the additional chamber or the additional chambers. The connection channel can comprise, for example, at least one section of the channel and/or the additional channel and/or the further channel.

For example, the various chambers can be acoustically tuned differently, so that each of the chambers damps a different frequency range of the sound.

### BRIEF DESCRIPTION OF THE DRAWINGS

Embodiments of the invention are described hereinafter with reference to the appended drawings. Neither the description nor the drawings are to be understood as a restriction of the scope of the invention. In the drawings:

FIG. 1 shows a respiratory therapy device according to one embodiment of the invention.

FIG. 2 shows a chamber of a respiratory therapy device according to one embodiment of the invention having an insert element.

FIG. 3 shows a chamber of a respiratory therapy device according to one embodiment of the invention having openings offset in relation to one another.

FIG. 4 shows a chamber of a respiratory therapy device according to one embodiment of the invention having a channel filled with filter material.

FIG. 5 shows a chamber of a respiratory therapy device according to one embodiment of the invention having two channels, each of which protrudes into the interior of the chamber.

FIG. 6 shows a chamber of a respiratory therapy device according to one embodiment of the invention having two channels, only one of which protrudes into the interior of the chamber.

FIG. 7 shows a chamber of a respiratory therapy device according to one embodiment of the invention having two channels, only one of which protrudes into the exterior surroundings of the chamber.

FIG. 8 shows two chambers of a respiratory therapy device connected to one another in series according to one embodiment of the invention having asymmetrical structure.

FIG. 9 shows two chambers of a respiratory therapy device connected to one another in series according to one embodiment of the invention having symmetrical structure.

The drawings are solely schematic and are not to scale. If identical reference signs are used in different drawings, these reference signs designate identical or identically-acting features.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

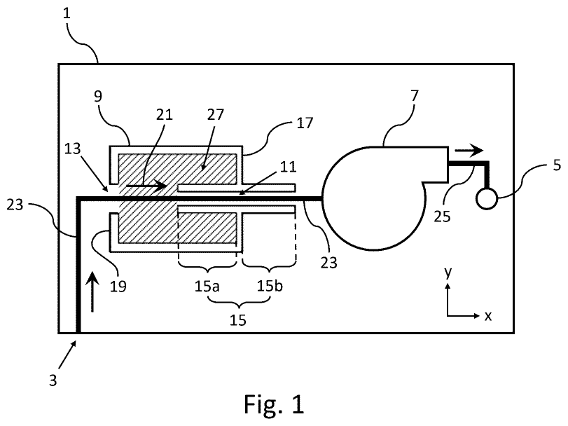

FIG. 1 shows a respiratory therapy device 1, which comprises a gas inlet 3, a gas outlet 5, a fan 7 for conveying respiratory gas from the gas inlet 3 to the gas outlet 5, and a chamber 9 for sound damping.

For example, the gas inlet 3 can be connectable to a suitable respiratory gas source. Alternatively or additionally, the gas outlet 5 can be connectable via a hose to a corresponding patient interface, for example, a nose mask, a facial mask, a (high-flow) nasal cannula, or a tube.

The chamber 9 comprises a first opening 11, a second opening 13, and a tubular channel 15. The channel 15 comprises in this example an inner channel section 15a protruding from the first opening 11 into the interior of the chamber 9 and an outer channel section 15b protruding from the first opening 11 into the exterior surroundings of the chamber 9.

The respiratory therapy device 1 is designed so that the respiratory gas, when it is conveyed from the gas inlet 3 to the gas outlet 5, flows between the first opening 11 and the second opening 13, here from the second opening 13 to the first opening 11, through the chamber 9 and passes the channel 15 in this case.

As shown by way of example in FIG. 1, the chamber 9 can be delimited in a longitudinal direction x on the one hand by a first wall 17 having the first opening 11 and on the other hand by a second wall 19 having the second opening 13. A distance between the two walls 17, 19 in the longitudinal direction x can correspond to a length of the chamber 9.

In this example, a first end of the inner channel section 15a is connected to the first wall 17, while a second unconnected end of the inner channel section 15a protrudes into the interior of the chamber 9. The second end of the inner channel section 15a can be open and can be partially or completely opposite to the second opening 13 in the second wall 19 viewed in the direction of a flow 21 of the respiratory gas from the second opening 13 to the first opening 11.

In a corresponding manner, a first end of the outer channel section 15b can be connected to the first wall 17, while a second end of the outer channel section 15b can protrude into the exterior surroundings of the chamber 9. The second end of the outer channel section 15b can be fluidically connected directly or indirectly to a (section-side) inlet of the fan 7. In this example, the second end is connected to the inlet by means of a first gas line 23. A (pressure-side) output of the fan 7 can be fluidically connected, for example, to the gas outlet 5 by means of a gas line 25.

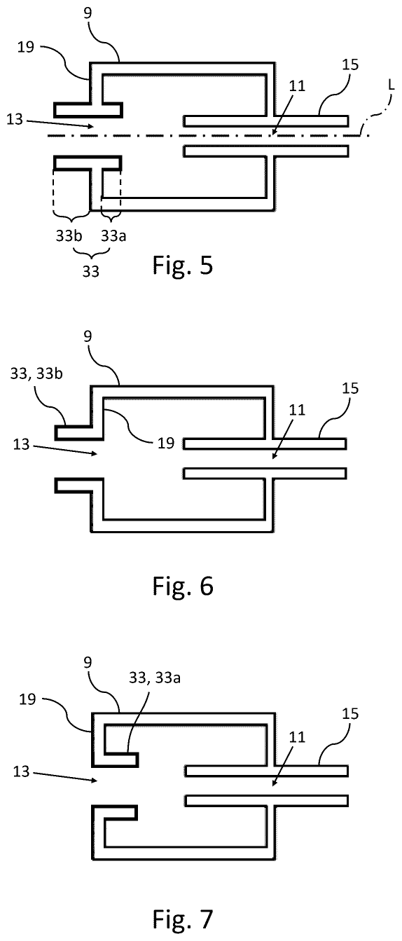

As can be seen in FIG. 1, the second opening 13 can be a simple passage through the second wall 19. A center axis of the second, for example, round opening 15 can lie on a longitudinal axis of the channel 15 (see also FIG. 5) or can be aligned parallel thereto (see also FIG. 3).

In addition, the chamber 9 can comprise a gas-permeable filter material 27 for filtering particles and/or moisture out of the respiratory gas. The filter material 27 can be arranged in a flow path between the first opening 11 and the second opening 13 in the interior of the chamber 9 and/or in the channel 15. The chamber 9 can be filled largely or even completely with the filter material 27. The channel 15 is free of any filter material 27 in FIG. 1.

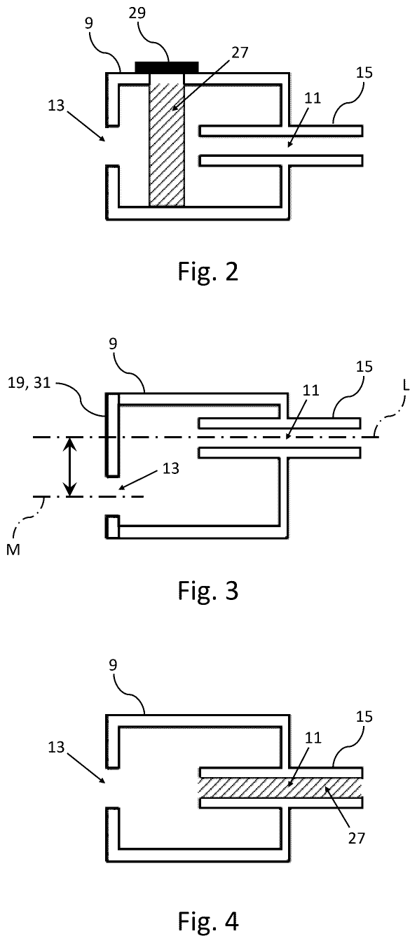

FIG. 2 shows an embodiment of the chamber 9 having an optional insert element 29, on which the filter material 27 is fastened. In this example, the insert element 29 is insertable into the interior of the chamber 9 in a height direction y orthogonal to the longitudinal direction x. This enables easy replacement of the filter material 27.

FIG. 3 shows an embodiment of the chamber 9 in which the longitudinal axis L of the channel 15 and the center axis M of the second opening 13 are offset in relation to one another in the height direction y (marked by a double arrow). The chamber 9 additionally comprises a removable chamber cover 31 here, which forms the second wall 19 when it encloses the chamber 9.

FIG. 4 shows an embodiment of the chamber 9 in which the filter material 27 is exclusively arranged in the channel 15. The filter material 27 can partially or, as here, completely fill the channel 15.

FIG. 5 shows an embodiment of the chamber 9 having a further channel 33, which can comprise a further inner channel section 33a protruding from the second wall 19 into the interior of the chamber 9 and/or a further outer channel section 33b protruding from the second wall 19 into the exterior surroundings of the chamber 9. The channel 15 and the further channel 33 can have a common longitudinal axis L. Moreover, the further channel 33 can have a significantly larger flow cross section than the channel 15. Depending on the flow direction of the respiratory gas, the further channel 33 can also have a significantly smaller flow cross section than the channel 15. Alternatively or additionally, the channel 15 and the further channel 33 can significantly deviate from one another in their length. For example, a length of the further channel 33 can be at most only a third of a length of the channel 15. Such a length ratio can contribute to reducing pressure losses and/or further improving the sound-damping effect of the chamber 9.

FIG. 6 shows an embodiment of the chamber 9 in which the further channel 33, in contrast to the embodiment of FIG. 5, only comprises the further outer channel section 33b. In this case, the second opening 13 can substantially terminate flush with an inner surface of the second wall 19 facing toward the interior of the chamber 9.

FIG. 7 shows an embodiment of the chamber 9 in which the further channel 33, in contrast to the embodiment of FIG. 5, only comprises the further inner channel section 33a. In this case, the second opening 13 can substantially terminate flush with an outer surface of the second wall 19 facing toward the exterior surroundings of the chamber 9.

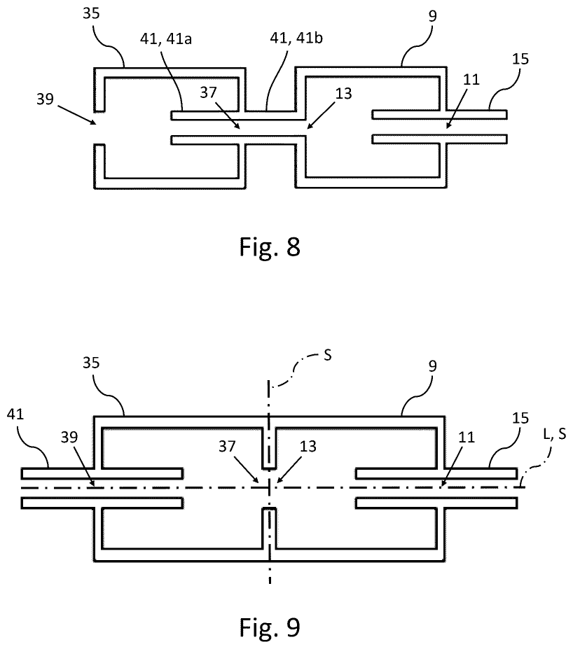

FIG. 8 shows an embodiment in which the chamber 9 is connected in series to an additional chamber 35 for sound damping. Similarly to the chamber 9, the additional chamber 35 can comprise an additional first opening 37, an additional second opening 39, and an additional tubular channel 41. The additional channel 41 can comprise an additional inner channel section 41a protruding from the additional first opening 37 into the interior of the additional chamber 35 and/or an additional outer channel section 41b protruding from the additional first opening 37 into the exterior surroundings of the additional chamber 35. The respiratory therapy device can be designed so that the respiratory gas, when it is conveyed from the gas inlet to the gas outlet, furthermore flows between the additional first opening 37 and the additional second opening 39, for example, from the additional second opening 39 to the additional first opening 37, through the additional chamber 35 and passes the additional channel 41 in this case.

In this example, the additional first opening 37 is fluidically connected via the additional outer channel section 41b to the second opening 13 of the chamber 9, so that the respiratory gas flows in succession through the chambers 9, 35 when it is conveyed from the gas inlet to the gas outlet.

In this way, the sound-damping effect can be further improved. For example, the various chambers 9, 35 can be acoustically tuned differently, so that each of the chambers 9, 35 damps a different frequency range of the sound.

The chamber 9 can also be connected in series to more than one additional chamber 35, for example, to at least two or at least four additional chambers 35.

FIG. 9 shows an embodiment in which the additional channel 41, in contrast to the embodiment of FIG. 8, is arranged at the additional second opening 39. In this case, the two chambers 9, 35 can be fluidically connected to one another via a simple passage in a common partition wall. The passage can form both the additional first opening 37 of the additional chamber 35 and the second opening 13 of the chamber 9. For example, the two chambers 9, 35 can be formed symmetrically with respect to an imaginary line of symmetry S in the vertical and/or horizontal direction.

Finally, it is to be noted that terms such as “has”, “comprises”, “includes”, “having” etc. do not exclude other elements or steps and indefinite articles such as “a” or “an” do not exclude a plurality.

Furthermore, it is to be noted that features or steps which are described with reference to one of the above embodiments can also be used in combination with features or steps which are described with reference to other ones of the above embodiments.

Reference signs in the claims are not to be understood as a restriction of the scope of the subject matter defined by the claims.

### LIST OF THE REFERENCE NUMERALS

1 respiratory therapy device
3 gas inlet
5 gas outlet
7 fan
9 chamber
11 first opening
13 second opening
15 tubular channel
15a inner channel section
15b outer channel section
17 first wall
19 second wall
21 flow
23 first gas line
25 second gas line
27 filter material
29 insert element
31 chamber cover
33 tubular further channel
33a further inner channel section
33b further outer channel section
35 additional chamber
37 additional first opening
39 additional second opening
41 additional tubular channel
41a additional inner channel section
41b additional outer channel section
x longitudinal direction
y height direction
L longitudinal axis
M center axis
S axis of symmetry

What is claimed is:

1. A respiratory therapy device, wherein the device comprises:
a gas inlet;
a gas outlet;
a fan for conveying respiratory gas from the gas inlet to the gas outlet;
a chamber for sound damping, the chamber comprising a first opening, a second opening, and a tubular channel, which comprises an inner channel section protruding from the first opening into an interior of the chamber and/or an outer channel section protruding from the first opening into exterior surroundings of the chamber;
the respiratory therapy device being configured so that respiratory gas, when it is conveyed from the gas inlet to the gas outlet, flows between the first opening and the second opening through the chamber and passes thetubular channel in this case.

2. The respiratory therapy device of claim 1,
wherein the second opening is at least partially opposite to the first opening and/or an open end of the inner channel section viewed in a direction of a flow of the respiratory gas through the chamber; and/or
wherein the second opening has a different flow cross section than the first opening and/or than an open end of the inner channel section; and/or
wherein the respiratory therapy device is configured so that the respiratory gas, when it is conveyed from the gas inlet to the gas outlet, flows from the second opening to the first opening through the chamber.

3. The respiratory therapy device of claim 2, wherein the second opening has a a larger flow cross section than the first opening and/or than an open end of the inner channel section.

4. The respiratory therapy device of claim 1,
wherein the second opening terminates flush with an inner surface of a wall of the chamber facing toward an interior of the chamber; and/or
wherein the second opening terminates flush with an outer surface of a wall of the chamber facing toward exterior surroundings of the chamber.

5. The respiratory therapy device of claim 1,
wherein a flow cross section of the inner channel section corresponds in its size and/or shape with a flow cross section of the outer channel section; and/or
wherein the inner channel section and the outer channel section have a common longitudinal axis.

6. The respiratory therapy device of claim 1,
wherein the chamber is delimited in a longitudinal direction on the one hand by a first wall and on the other hand by a second wall, wherein the first wall comprises the first opening and/or the second wall comprises the second opening.

7. The respiratory therapy device of claim 6,
wherein the inner channel section protrudes into the interior of the chamber at most up to half of a length of the chamber in a longitudinal direction between the first wall and the second wall; and/or
wherein a first end of the inner channel section is connected to the first wall and a second unconnected end of the inner channel section protrudes into an interior of the chamber.

8. The respiratory therapy device of claim 1,
wherein the chamber further comprises a removable and/or movably mounted chamber cover for closing the chamber.

9. The respiratory therapy device of claim 8,
wherein the chamber cover, when it encloses the chamber, at least forms a section of the first wall and/or the second wall.

10. The respiratory therapy device of claim 1,
wherein the chamber further comprises a filter material for filtering particles and/or moisture from the respiratory gas flowing through the chamber between the first opening and the second opening.

11. The respiratory therapy device of claim 10,
wherein the filter material at least partially fills an interior of the chamber and/or at least partially fills the channel; and/or
wherein the filter material is arranged opposite to the first opening and/or the second opening; and/or
wherein the filter material is formed as a part of an insert element insertable into the chamber and/or the channel.

12. The respiratory therapy device of claim 11,
wherein the filter material is fastened on a chamber cover and is removable and/or movably mounted together with the chamber cover.

13. The respiratory therapy device of claim 1,
wherein the chamber further comprises a tubular further channel, wherein the further channel comprises a further inner channel section protruding from the second opening into an interior of the chamber and/or a further outer channel section protruding from the second opening into exterior surroundings of the chamber;
wherein the respiratory therapy device is configured so that the respiratory gas, when it is conveyed from the gas inlet to the gas outlet, furthermore passes the further channel.

14. The respiratory therapy device of claim 13,
wherein an open end of the further inner channel section is at least partially opposite to the first opening and/or an open end of the inner channel section viewed in a direction of a flow of the respiratory gas through the chamber; and/or
wherein an open end of the further inner channel cross section has a different flow cross section than the first opening and/or than an open end of the inner channel section; and/or
wherein a length of the further inner channel section is at most a third of a length of the inner channel section; and/or
wherein the further inner channel section and the further outer channel section have a common longitudinal axis; and/or
wherein a flow cross section of the further inner channel section corresponds in its size and/or shape with a flow cross section of the further outer channel section.

15. The respiratory therapy device of claim 14, wherein the open end of the further inner channel cross section has a larger flow cross section than the first opening and/or than an open end of the inner channel section.

16. The respiratory therapy device of claim 13,
wherein the channel has a different flow cross section than the further channel; and/or
wherein a length of the further channel is at most a third of a length of the channel; and/or
wherein the channel and the further channel have a common longitudinal axis.

17. The respiratory therapy device of claim 16, wherein the channel has a smaller flow cross section than the further channel.

18. The respiratory therapy device of claim 1, wherein the device further comprises:
an additional chamber for sound damping. Which comprises an additional first opening, an additional second opening, and an additional tubular channel, the additional channel comprising an additional inner channel section protruding from the additional first opening into an interior of the additional chamber and/or an additional outer channel section protruding from the additional first opening into exterior surroundings of the additional chamber;
the respiratory therapy device being configured so that the respiratory gas, when it is conveyed from the gas inlet to the gas outlet, furthermore flows between the additional first opening and the additional second opening through the additional chamber and passes the additional channel in this case.
